(19) United States
(12) Patent Application Publication
(10) Pub. No.: US 2026/0232461 A1
(43) Pub. Date: Aug. 13, 2026

(54) Insertion Tool for Total Ankle Implants

(71) Applicant: Arthrex, Inc., Naples, FL (US)

(72) Inventor: Scott Smyth, Naples, FL (US)

(21) Appl. No.: 19/533,489

(22) Filed: Feb. 9, 2026

Related U.S. Application Data
(60) Provisional application No. 63/757,285, filed on Feb. 11, 2025.
(60) Provisional application No. 63/756,459, filed on Feb. 10, 2025.

(51) Int. Cl. A61F 2/46; A61F 2/42; A61B 17/56

(52) U.S. Cl. CPC A61F 2/4606; A61F 2/4202; A61B 2017/567; A61F 2002/4622; A61F 2002/4628; A61F 2002/4631

(57) ABSTRACT

The present disclosure provides a surgical insertion tool including a first arm, a second arm, and a flexible crossbar configured to couple the first arm to the second arm. The first arm includes a proximal end and a distal end. The proximal end of the first arm has a handle and the distal end has an implant interface configured to hold a portion of an implant. The second arm includes a proximal end and a distal end. The proximal end of the second arm has a handle and the distal end has an implant interface configured to hold a portion of an implant. The flexible crossbar includes a first end coupled at an intermediate point between the proximal end and the distal end of the first arm and a second end coupled at an intermediate point between the proximal end and the distal end of the second arm.

### CROSS-REFERENCE TO RELATED APPLICATIONS

This application claims priority to (i) U.S. Provisional Application No. 63/756,459 entitled “Insertion Tool for Total Ankle Implants,” filed on February 10, 2025, and (ii) U.S. Provisional Application No. 63/757,285 entitled “Insertion Tool for Total Ankle Implants,” filed on February 11, 2025, the contents of each of which are hereby incorporated by reference in their entirety.

### BACKGROUND

Total ankle replacement procedures demand precise placement of the talus implant to ensure optimal functionality and patient outcomes. However, conventional methods pose significant challenges. Surgeons must manually handle and balance the implant during insertion, often while applying bone cement and ensuring alignment, making the process both complex and error-prone. This approach introduces several potential drawbacks, including (i) a risk of contamination, as manual handling may compromise sterility when gloves contact critical implant surfaces, (ii) limited stability, due to the lack of dedicated tools, which complicates maintaining control and proper orientation, (iii) suboptimal cement application, as instability can lead to uneven cement distribution or premature curing, and (iv) a risk of misalignment, as the absence of specialized tools extends surgical workflows and increases the likelihood of positional errors.

Disclosed herein are improved surgical insertion tools for use in such total ankle replacement procedures.

### SUMMARY

The present disclosure relates to surgical insertion tools for total ankle replacement procedures. An example surgical insertion tool for total ankle replacement procedures, described herein, is configured to securely grip an implant, allowing a surgeon to handle it with stability during insertion into a patient’s bone.

In one aspect, a surgical insertion tool is provided. The surgical insertion tool includes a first arm having a proximal end and a distal end. The proximal end of the first arm has a handle and the distal end of the first arm has an implant interface configured to hold a portion of an implant. The surgical insertion tool also includes a second arm having a proximal end and a distal end. The proximal end of the second arm has a handle and the distal end of the second arm has an implant interface configured to hold a portion of an implant. Additionally, the surgical insertion tool includes a flexible crossbar configured to couple the first arm to the second arm. The flexible crossbar includes a first end coupled at an intermediate point between the proximal end and the distal end of the first arm and a second end coupled at an intermediate point between the proximal end and the distal end of the second arm.

In another aspect, a surgical insertion tool is provided. The surgical insertion tool includes a first arm having a proximal end, a distal end opposite the proximal end, and an inner side surface extending from the proximal end to the distal end. The proximal end of the first arm has a pair of holes disposed on the inner side surface that extend into the first arm. The distal end of the first arm has a first implant interface configured to interface with a portion of an implant. The surgical insertion tool also includes a second arm having a proximal end, a distal end opposite the proximal end, and an inner side surface extending from the proximal end to the distal end. The proximal end of the second arm has a pair of protrusions extending horizontally from the inner side surface and is configured to be inserted into the corresponding pair of protrusions of the first arm to connect the first arm to the second arm. The distal end of the second arm has a second implant interface configured to interface with a portion of an implant. The surgical insertion tool also includes a flexible component configured to couple the proximal end of the first arm to the proximal end of the second arm.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates a first example of a surgical insertion tool.

FIG. 2 illustrates an example implementation of the surgical insertion tool of FIG. 1 securing an implant.

FIG. 3 illustrates a second example of a surgical insertion tool.

FIG. 4 illustrates a third example of a surgical insertion tool.

FIG. 5 illustrates an example implementation of the surgical insertion tool of FIG. 4 securing an implant.

FIG. 6 illustrates an exploded view of a fourth example of a surgical insertion tool.

FIG. 7A illustrates a first arm of the surgical insertion tool of FIG. 6.

FIG. 7B illustrates a second arm of the surgical insertion tool of FIG. 6.

### DETAILED DESCRIPTION

As discussed above, the process of manually handling and balancing an implant during insertion—while simultaneously applying bone cement and ensuring proper alignment with the bone—is both complex and prone to errors. The intricate coordination required in such procedures increases the risk of complications, making it essential to develop tools that simplify and optimize the process.

To address these challenges, a surgical insertion tool is provided to improve precision, efficiency, and overall surgical outcomes. The tool is configured to reduce contamination by eliminating the need for direct contact between gloves and the implant. The tool minimizes the likelihood of introducing contaminants into the surgical site, thereby enhancing patient safety.

Additionally, the tool provides improved control through its gripping mechanism, which ensures precise and stable handling of the implant. For example, the tool allows surgeons to manipulate the implant confidently, even in challenging surgical environments, such as within the joint, where maintaining accuracy is critical.

Additionally, the tool optimizes cement application. By securely holding the implant in place, the tool frees the surgeon’s hands to focus on applying bone cement evenly and efficiently. This not only enhances the quality of the cementing process but also reduces the risk of improper fixation.

Finally, the tool streamlines the surgical workflow by replacing manual insertion with a guided, tool-assisted approach. This reduces procedural complexity, saves time, and contributes to more predictable and successful surgical outcomes. Together, these features address the limitations of manual handling and elevate the standard of care in implant insertion procedures.

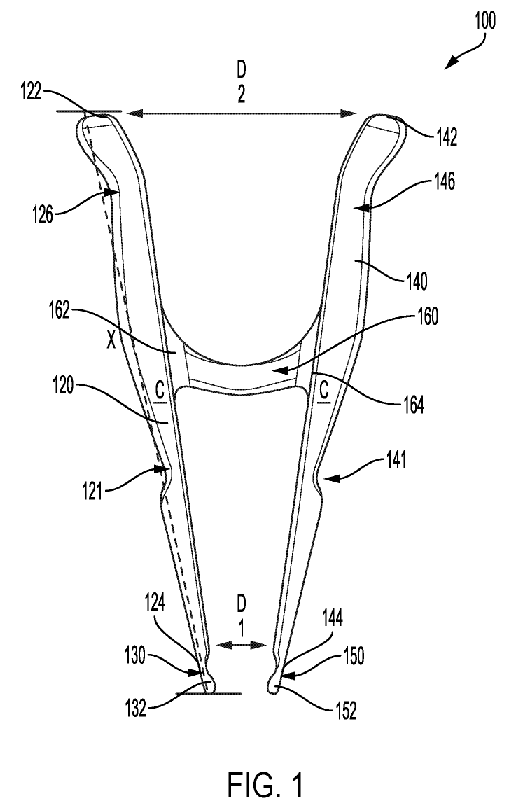

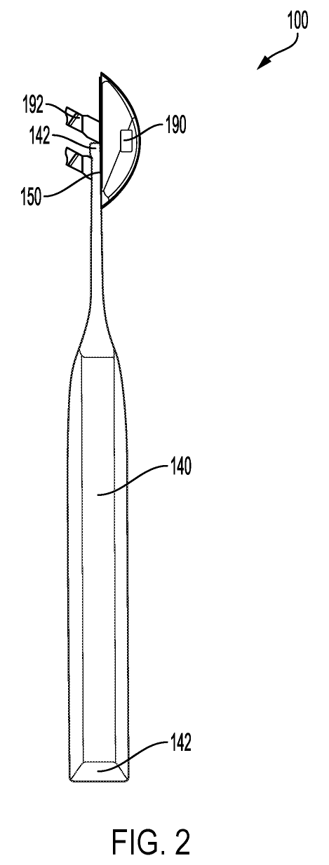

With reference to FIGS. 1 and 2, FIG. 1 illustrates a first example of a surgical insertion tool 100. FIG. 2 illustrates an example implementation of the surgical insertion tool 100 securing an implant.

The surgical insertion tool 100 includes a first arm 120, a second arm 140, and a flexible crossbar 160 configured to couple the first arm 120 to the second arm 140. The first arm 120 and the second arm 140 include similar components as described below.

The first arm 120 includes a proximal end 122 and a distal end 124 that is opposite the proximal end 122. The proximal end 122 of the first arm 120 includes a first handle 126 and the distal end 124 of the first arm 120 includes a first implant interface 130.

The first handle 126 is ergonomically configured to fit the natural contour of a hand, including a curved, elongated profile that tapers slightly toward the middle for a secure grip. In some examples, the first handle 126 may include a plurality of ridges along its surface to enhance grip and prevent slipping during use, even in wet or gloved conditions. For example, with evenly spaced ridges running perpendicular to the length of the first handle 126, additional friction and control for precise handling may be provided.

The first implant interface 130 includes a first groove 132 configured to receive a portion of the implant. In this example, the first groove 132 is configured to match the profile of a talar peg of a talus implant, as is depicted in FIG. 2. In some examples, the first groove 132 may be shaped to match the profile of the implant's body, ensuring a complementary fit. In other examples, the first groove 132 may also include a friction surface. The friction surface may be made of rubber material to help improve the grip on the implant.

The first arm 120 also includes a first notch 121 disposed adjacent to the distal end 124. In particular, the first notch 121 is positioned between a center point C of the first arm 120 and the distal end 124. In some examples, the first notch 121 is positioned closer to the center point C than the distal end 124. Additionally, the first notch 121 is configured to receive a finger of a surgeon. For example, the first notch 121 is configured to indicate to the surgeon where to manipulate/hold the first arm 120 and provide improved grip and control during manipulation of the first arm 120 to secure an implant.

Similar to the first arm 120, the second arm 140 includes a proximal end 142 and a distal end 144 opposite the proximal end 142. The proximal end 142 of the second arm 140 has a second handle 146, and the distal end 144 has a second implant interface 150.

The second handle 146 is similar to the first handle 126 described above. The second handle 146 is ergonomically configured to fit the natural contour of a hand, including a curved, elongated profile that tapers slightly toward the middle for a secure grip.

The second implant interface 150 is also similar to the first implant interface 130. The second implant interface includes a second groove 152 configured to receive a portion of the implant. Like the first groove 132, the second groove 152 may be shaped to match the profile of the implant's body or peg, ensuring a complementary fit.

As depicted in FIG. 1, the second arm 140 also includes a second notch 141 that is positioned between a center point C of the second arm 140 and the distal end 144. Similar to the first notch 121, the second notch 141 is configured to receive a finger of the surgeon.

Each of the first arm 120 and the second arm 140 extend a length X from corresponding proximal ends 122, 142 to corresponding distal ends 124, 144. The length X may be approximately at least 10 centimeters (cm), approximately at least 15 cm, approximately at least 20 cm, or approximately at least 25 cm.

Additionally, each arm (i.e., the first arm 120, the second arm 140) is shaped to include a wide proximal end (i.e., proximal end 122, proximal end 142), which gradually narrows as it extends toward the distal end (i.e., distal end 124, distal end 144). This tapering profile enhances maneuverability by reducing bulk, allowing the surgical insertion tool 100 to access confined or intricate surgical spaces with ease. At the distal ends (i.e., distal end 124, distal end 144), the arms (i.e., the first arm 120, the second arm 140) are further refined into tapered tips with a narrow, pointed shape for securely gripping and stabilizing implants during insertion.

As noted above, the surgical insertion tool 100 includes the flexible crossbar 160, which is configured to connect the first arm 120 to the second arm 140, and allow the surgical insertion tool 100 to bend/flex when actuated. The flexible crossbar 160 includes a first end 162 and a second end 164 that is opposite the first end 162. The flexible crossbar 160 is positioned between the proximal ends (i.e., proximal end 122, proximal end 142), and the distal ends (i.e., distal end 124, distal end 144) of the first arm 120 and the second arm 140.

As depicted in FIG. 1, the first end 162 of the flexible crossbar 160 is coupled to the first arm 120 at an intermediate point located between the center point C of the first arm 120 and the proximal end 122 of the first arm 120. Similarly, the second end 164 of the flexible crossbar 160 is coupled to the second arm 140 at an intermediate point located between the center point C of the second arm 140 and the proximal end 142 of the second arm 140.

In some examples, the first end 162 of the flexible crossbar 160 is coupled to the first arm 120 at the center point C of the first arm 120 and the second end 164 of the flexible crossbar 160 is coupled to the second arm 140 at the center point C of the second arm 140.

The flexible crossbar 160 biases the distal end 124 of the first arm 120 a first distance D1 away from the distal end 144 of the second arm 140, and biases the proximal end 122 of the first arm 120 a second distance D2 away from the proximal end 142 of the second arm 140. In this configuration, the first distance D1 is smaller than the second distance D2, such that the first and second arms 120 and 140, connected by the flexible crossbar 160, are positioned at an angle to one another. For example, the flexible crossbar 160 may be under tension or pre-shaped to pull the first and second arms 120 and 140 inward at the distal ends 124, 144, while allowing the proximal ends 122, 142 to remain further apart. In some examples, the overall configuration of the surgical insertion tool 100, with the first arm 120 and second arm 140 connected by the flexible crossbar 160, resembles an “H” shape.

As depicted in FIG. 1, the first arm 120, the second arm 140, and the flexible crossbar 160 are manufactured as a single, integrated part. In such an example, the flexible crossbar 160 is a single component with its material and structural properties configured to provide the required flexibility and tension control. In some examples, the first arm 120, the second arm 140, and the flexible crossbar 160 may be separate components that are coupled together to form the surgical insertion tool 100.

In some examples, each of the first arm 120, second arm 140, and the flexible crossbar 160 may be made of either metal or a durable, lightweight composite material that offers both strength and flexibility. Such materials may include stainless steel, titanium, aluminum alloys, or Nitinol for metals, and polymer composites, carbon fiber reinforced plastics (CFRP), or advanced polymers for lightweight composites.

In an example implementation, the surgical insertion tool 100 is configured to securely hold an implant 190. To open or separate the distal ends 124 and 144 from each other, the proximal ends 122 and 142 are actuated via first and second handles 126 and 146. When the first and second handles 126 and 146 are compressed toward each other, the distal ends 124 and 144 move apart, creating an opening to accommodate the implant 190. Conversely, releasing the first and second handles 126 and 146 allows the surgical insertion tool 100 to apply a secure gripping force on the implant 190, ensuring stability during the surgical procedure.

As depicted in FIG. 2, the implant 190 is secured between the first implant interface 130 and the second implant interface 150. Specifically, the first groove 132 and the second groove 152 receive a corresponding talar peg 192 of the implant 190. The grooves (i.e., first groove 132 and second groove 152) are shaped to complement the profile of the talar peg 192, ensuring a secure fit. Additionally, in some examples, each groove (i.e., first groove 132, second groove 152) may include a friction surface, which can be made from a rubber material to enhance the grip on the implant further.

In certain examples, to further stabilize the implant 190 and provide enhanced control during insertion into the patient, the surgeon may utilize the first notch 121 and the second notch 141. The surgeon may actuate the first arm 120 and the second arm 140 by compressing both arms together at the first notch 121 and the second notch 141. As a result, a greater gripping force on the implant 190 is provided. By leveraging the notches 121 and 141, the surgeon can maintain better control of the implant’s positioning, minimizing movement and ensuring accurate placement during the procedure.

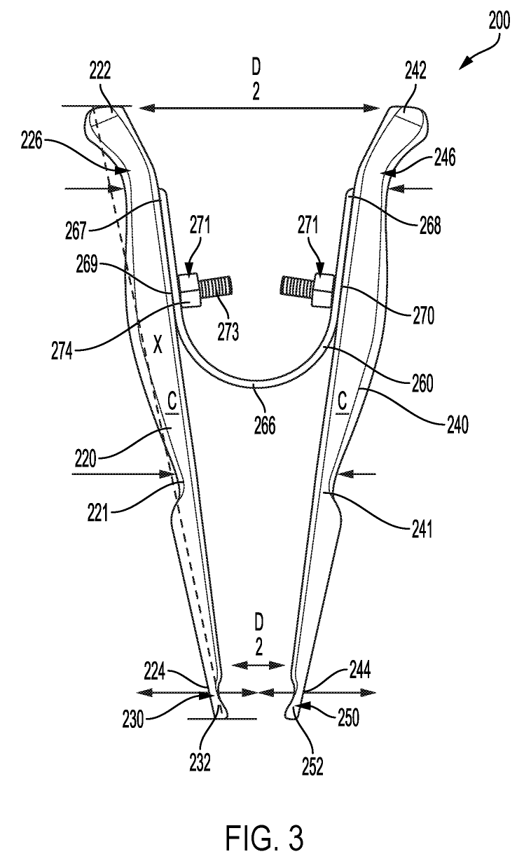

FIG. 3 illustrates a second example of a surgical insertion tool 200, where the surgical insertion tool 200 includes another example of a flexible crossbar 260. In such an example, one or more components of the surgical insertion tool 200 are the same or similar in form and function to one or more components of the surgical insertion tool 100.

Referring to FIG. 3, similar to the surgical insertion tool 100, the surgical insertion tool 200 includes a first arm 220, a second arm 240, and a flexible crossbar 260 configured to couple the first arm 220 to the second arm 240. In this example, the first arm 220, the second arm 240, and the flexible crossbar 260 are separate components that are coupled to form the surgical insertion tool 200.

As shown in FIG. 3, each of the first arm 220 and the second arm 240 includes a proximal end 222, 242 and a distal end 224, 244 opposite the proximal end 222, 242. In particular, the first arm 220 includes proximal end 222 and distal end 224, and the second arm 240 includes proximal end 242 and distal end 244. Each of the first arm 220 and the second arm 240 also includes a handle (i.e., a first handle 226, a second handle 246) positioned at the corresponding proximal end 222, 242 and an implant interface (i.e., a first implant interface 230, a second implant interface 250) positioned at the corresponding distal end 224, 244.

Similar to the surgical insertion tool 100, each of the first handle 226 and the second handle 246 of the surgical insertion tool 200 is ergonomically formed to fit the natural contour of a hand, featuring a curved, elongated profile that gently tapers toward the middle for a secure grip. Additionally, each of the first implant interface 230 and the second implant interface 250 includes a groove (i.e., a first groove 232, a second groove 252) configured to receive a portion of an implant. Each groove (i.e., the first groove 232, the second groove 252) is shaped to complement the profile of the implant's body or peg, ensuring a secure fit.

Furthermore, each of the first arm 220 and the second arm 240 includes a notch (i.e., a first notch 221 located near the distal end 224, a second notch 241 positioned near the distal end 244). Specifically, each notch is positioned between a center point C of the first arm 220 and the distal end 224. For example, the first notch 221 is positioned between the center point C of the first arm 220 and the distal end 224, and the second notch 241 is positioned between the center point C of the second arm 240 and the distal end 244. Each notch is configured to receive a finger of the surgeon, providing an intuitive point of manipulation.

The surgical insertion tool 200 also includes the flexible crossbar 260. However, unlike the flexible crossbar 160, the flexible crossbar 260 is configured to couple the first arm 220 to the second arm 240 via a plurality of fastening members 271, and is configured to control an amount of flexion via the plurality of fastening members 271.

For example, as depicted in FIG. 3, the flexible crossbar 260 includes a beam 266 with a first end 267, a second end 268, a first hole 269, a second hole 270, and fastening members 271. The first hole 269 is positioned near, but not directly at, the first end 267 of the beam 266. Similarly, the second hole 270 is positioned near, but not directly at, the second end 268 of the beam 266. Thus, a portion of the beam 266 extends beyond each hole (i.e., first hole 269, second hole 270, allowing the fastening members 271 to securely attach the crossbar 260 to the arms 220 and 240.

The beam 266 is coupled via the fastening members 271 at the first hole 269 and second hole 270, which are located along the length of the beam 266. The fastening members 271 couple the beam 266 to the first arm 220 at a point between the center point C of the first arm 220 and the proximal end 222, and to the second arm 240 at a point between the center point C of the second arm 240 and the proximal end 242. The configuration allows the flexible crossbar 260 to securely join the first and second arms 220, 240 while maintaining controlled flexibility or tension.

Each fastening member 271 includes a bolt 273 and a nut 274 for coupling the beam 266 to the first and second arms 220, 240, through corresponding holes (i.e., first hole 269, second hole 270). The fastening members 271 are configured to control an amount of flexion in the beam 266 by adjusting the tightness of the corresponding nuts 274 relative to the corresponding bolts 273. This adjustability enables the surgeon to fine-tune the tension in the flexible crossbar 260, which is essential for achieving the desired angle or compression between the first and second arms 220, 240 based on the specific requirements of the surgical insertion tool 200.

In some examples, the flexible crossbar 260 is configured to control an amount of flexion of the first arm 220 and the second arm 240 by adjusting the amount of tension applied through the fastening members 271. In particular, the first arm 220 and the second arm 240 can flex independently of each other, with the degree of flexion determined by adjusting the tightness of the nuts 274 relative to the bolts 273 of each respective fastening member 271.

In some examples, the flexible crossbar 260 is configured to control an amount of flexion between the first arm 220 and the second arm 240 by adjusting the amount of tension applied through the fastening members 271. This configuration allows the relative movement or flexion of the two arms 220, 240 as a unified unit to be precise. In particular, the first arm 220 and the second arm 240 can flex as a unit, with the degree of combined flexion controlled by adjusting the tightness of the nuts 274 relative to the bolts 273 of their respective fastening members 271.

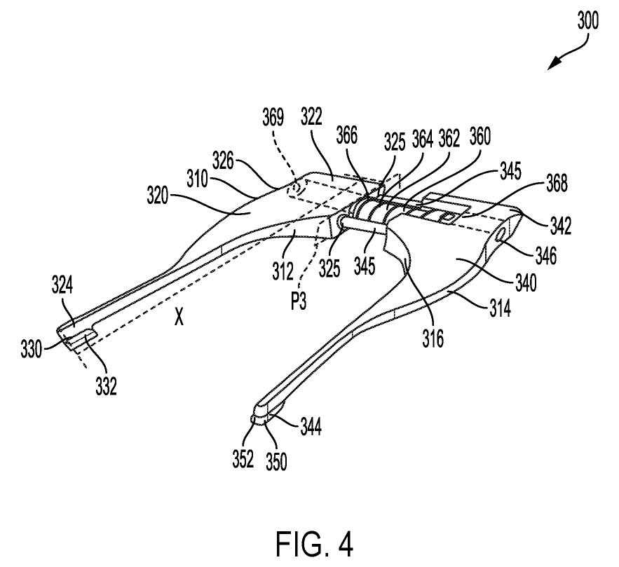

FIG. 4 illustrates a third example of a surgical insertion tool 300. The surgical insertion tool 300 includes a first arm 320, a second arm 340, and a flexible component 360 configured to couple the first arm 320 to the second arm 340.

The first arm 320 includes a proximal end 322 and a distal end 324, positioned opposite the proximal end 322. The first arm 320 further includes a first exterior side surface 310 extending continuously from the proximal end 322 to the distal end 324, as well as a first interior side surface 312 positioned opposite the first exterior side surface 310 and also extending from the proximal end 322 to the distal end 324.

The first arm 320 extends a length X from corresponding proximal end 322 to corresponding distal end 324. The length X may be approximately at least 10 centimeters (cm), approximately at least 15 cm, approximately at least 20 cm, or approximately at least 25 cm.

As depicted in FIG. 4, the first arm 320 includes a wide proximal end 322, which gradually narrows as it extends toward the distal end 324. This tapering profile enhances maneuverability by reducing bulk, allowing the tool to access confined or intricate surgical spaces with ease. In some examples, at the distal end 324, the first arm 320 is further refined into tapered tips with a narrow, pointed shape, which is ideal for securely gripping and stabilizing implants during insertion.

The proximal end 322 of the first arm 320 includes a pair of holes 325 positioned on the first interior side surface 312 of the first arm 320. Each hole 325 extends into the first arm 320 and is configured to receive a corresponding protrusion 345 extending from the second arm 340, as is further detailed below.

As shown in FIG. 4, each hole 325 extends perpendicular to a plane P3, which is parallel to the first interior side surface 312 of the first arm 320. This configuration ensures that the protrusions 345 from the second arm 340 align precisely with the holes 325, enabling secure coupling between the two arms 320 and 340.

In this configuration, the holes 325 are positioned on top of one another along the length of the first arm 320. However, holes 325 may be positioned in any configuration and are not limited to the present disclosure.

The proximal end 322 of the first arm 320 also includes a first handle 326 that is ergonomically configured to fit the natural contour of a hand, including a curved, elongated profile that tapers slightly toward the middle for a secure grip. In some examples, the first handle 326 may include a plurality of ridges along its surface to enhance grip and prevent slipping during use, even in wet or gloved conditions. For example, with evenly spaced ridges running perpendicular to the length of the first handle 326, additional friction and control for precise handling may be provided.

The distal end 324 of the first arm 320 includes a first implant interface 330. The first implant interface 330 includes a prong 332 configured to receive a portion of an implant. For instance, the prong 332 may be shaped to match the profile of the implant's body, ensuring a complementary fit. In some examples, the prong 332 may also include a friction surface. The friction surface may be made of rubber material to help improve the grip on the implant or have a textured pattern.

As noted above, the surgical insertion tool 300 includes the second arm 340. Similar to the first arm 320, the second arm 340 includes a proximal end 342 and a distal end 344, positioned opposite the proximal end 342. The second arm 340 further includes a second exterior side surface 314 extending continuously from the proximal end 342 to the distal end 344, as well as a second interior side surface 316 positioned opposite the second exterior side surface 314 and also extending from the proximal end 342 to the distal end 344.

The second arm 340 extends the same length as the first arm 320 and includes a similar shape and profile as the first arm 320. For example, as depicted in FIG. 4, the second arm 340 includes a wide proximal end 342, which gradually narrows as it extends toward the distal end 344.

The proximal end 342 of the second arm 340 includes a pair of protrusions 345 extending horizontally from the second interior side surface 316. The pair of protrusions 345 are configured to be inserted into the corresponding holes 325 disposed into the first arm 320. The pair of protrusions 345 extend perpendicularly from the plane P3 that is also parallel to the second interior side surface 316. The pair of protrusions 345 are configured to fit securely into corresponding holes 325 disposed on the first interior side surface 312 of the first arm 320. As a result, when the first arm 320 is connected to the second arm 340, the protrusions 345 are inserted into the holes 325, providing rotational stability and securely joining the two arms.

Similar to the first arm 320, the proximal end 342 of the second arm 340 also includes a second handle 346 that is ergonomically configured to fit the natural contour of a hand, including a curved, elongated profile that tapers slightly toward the middle for a secure grip.

Additionally, similar to the first arm 320, the distal end 344 of the second arm 340 includes a second implant interface 350. The second implant interface 350 includes a prong 352 configured to receive a portion of an implant. For instance, the prong 352 may be shaped to match the profile of the implant's body, ensuring a complementary fit.

As noted above, the surgical insertion tool 300 includes the flexible component 360 configured to couple the first arm 320 to the second arm 340. The flexible component 360 is configured to couple the proximal end 322 of the first arm 320 to the proximal end 342 of the second arm 340. The flexible component is also configured to bias the first arm 320 and the second arm 340 into an open position.

In some examples, the flexible component 360 includes a pin 362 and a helical compression spring 364 positioned around the pin 362. The pin 362 includes a first end 366 positioned within the first arm 320, near the proximal end 322, and a second end 368 positioned within the second arm 340, near the proximal end 342.

The spring 364 biases the first arm 320 and the second arm 340 outward. The ends of the spring 364 are securely seated within designated recesses or grooves in the first arm 320 and the second arm 340, preventing unwanted movement or misalignment. Thus, when the spring 364 is compressed, each end exerts a restorative force directed outward, pushing against the recesses or grooves in which they are seated. This force ensures that the arms 320 and 340 return to their original position when external pressure is released.

Additionally, the first end 366 of the pin 362 includes a knob 369 configured to lock the surgical insertion tool 300 in place, providing secure stabilization of the implant during manipulation. The knob 369 can be actuated (e.g., turned) to transition between a locking state, securing the tool, and an unlocked state, allowing for release and adjustment. For example, the knob 369 prevents the first arm 320 from moving relative to the second arm 340 by engaging a locking mechanism, such as a pin or latch,that secures both arms 320 and 340 in place.

In some examples, the flexible component 360 is a tension spring.

In an example implementation, the surgical insertion tool 300 is configured as a single-piece construction, such that all components are permanently connected. In yet another example implantation, the surgical insertion tool 300 is configured as a multi-piece construction, where the surgical insertion tool 300 can be disassembled.

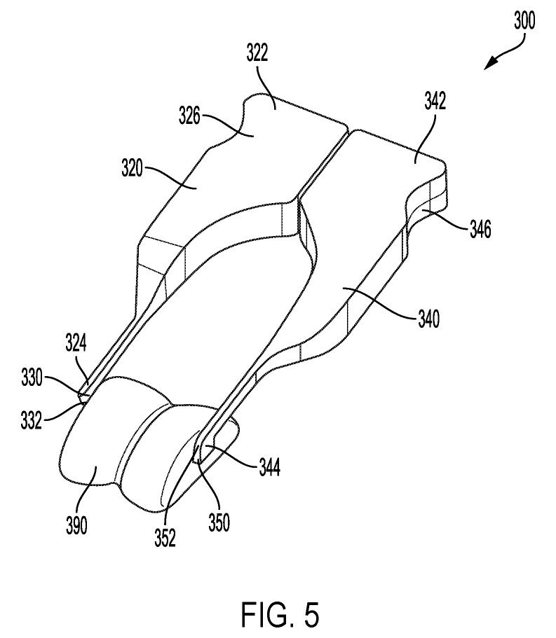

FIG. 5 illustrates an example implementation of the surgical insertion tool 300 securing an implant 390. To secure the implant 390, a surgeon actuates the distal end 324 of the first arm 320 and the distal end 344 of the second arm 340. When the distal end 324 of the first arm 320 and the distal end 344 of the second arm 340 are compressed toward each other a secure gripping force is applied on the implant 390. During this process, the flexible component 360 is tensioned, providing resistance and contributing to the controlled application of force on the implant 390. The flexible component 360’s tensioning mechanism also allows for precise adjustments in the grip, accommodating slight variations in the implant’s dimensions or positioning.

Once the distal end 324 of the first arm 320 and the distal end 344 of the second arm 340 are no longer actuated by the surgeon, the flexible component 360 biases the arms into their original open position. This biasing action releases the grip on the implant 390, allowing for easy removal or repositioning of the surgical insertion tool 300.

As depicted in FIG. 5, the implant 390 is securely held between the first implant interface 330 and the second implant interface 350. Specifically, the prong 332 and the prong 352 are configured to receive a body of the implant 390. The prong 332 and the prong 352 are contoured to complement the profile of the body of the implant 390, ensuring a snug and stable fit that minimizes unwanted movement during the procedure.

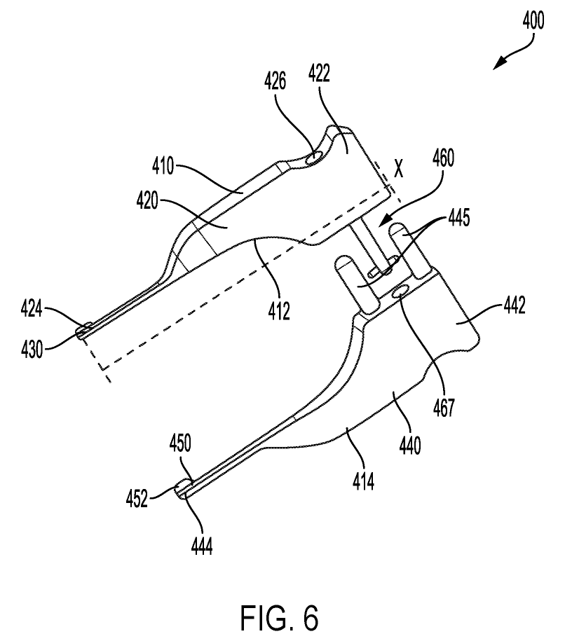

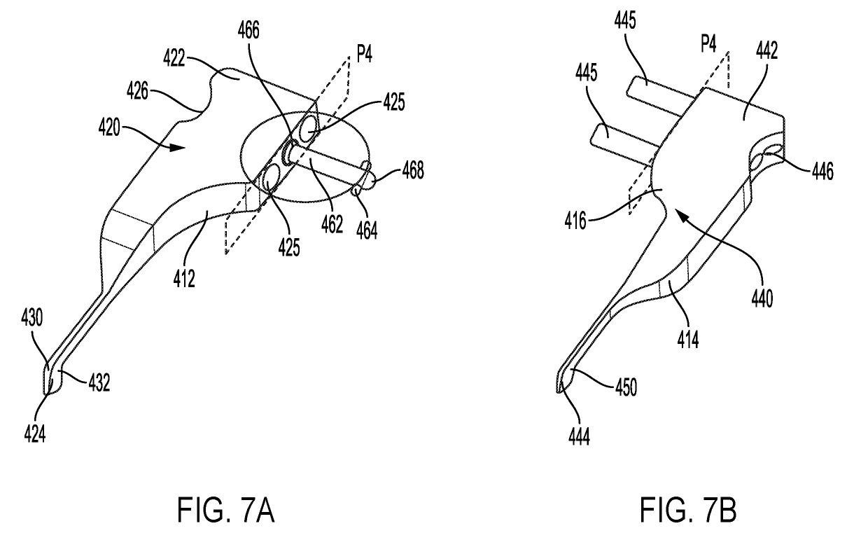

FIGS. 6, 7A, and 7B illustrate a fourth example of a surgical insertion tool 400. FIG. 6 illustrates an exploded view of the surgical insertion tool 400. FIG. 7A illustrates a first arm 420 of the surgical insertion tool 400. FIG. 7B illustrates a second arm 440 of the surgical insertion tool 400. In such an example, one or more components of the surgical insertion tool 400 are the same or similar in form and function to one or more components of the surgical insertion tool 300.

Referring to FIGS. 6, 7A, and 7B, the surgical insertion tool 400 includes a first arm 420, a second arm 440, and a flexible component 460 configured to couple the first arm 420 to the second arm 440.

Each of the first arm 420 and the second arm 440 includes a proximal end 422, 442 and a distal end 424, 444 opposite the proximal end 422, 442. In particular, the first arm 420 includes the first proximal end 422 and the first distal end 424, and the second arm 440 includes the second proximal end 442 and the second distal end 444.

Each of the first arm 420 and the second arm 440 include an interior and an exterior side surface. The first arm 420 includes a first exterior side surface 410 extending continuously from the proximal end 422 to the distal end 424, as well as a first interior side surface 412 positioned opposite the first exterior side surface 410 and also extending from the proximal end 422 to the distal end 424. The second arm 440 includes a second exterior side surface 414 extending continuously from the proximal end 442 to the distal end 444, as well as a second interior side surface 416 positioned opposite the second exterior side surface 414 and also extending from the proximal end 442 to the distal end 444.

Each of the first arm 420 and the second arm 440 extends a length X from corresponding proximal end (i.e., proximal end 422, proximal end 442) to corresponding distal end (i.e., distal end 424, distal end 444). The length X may be approximately at least 10 centimeters (cm), approximately at least 15 cm, approximately at least 20 cm, or approximately at least 25 cm.

As depicted in FIG. 4, each of the first arm 420 and the second arm 440 includes a wide proximal end (i.e., proximal end 422, proximal end 442), which gradually narrows as it extends toward the (i.e., distal end 424, distal end 444). This tapering profile enhances maneuverability by reducing bulk, allowing the tool to access confined or intricate surgical spaces with ease. In some examples, at the distal end (i.e., distal end 424, distal end 444), each arm (i.e., 420, 440) is further refined into tapered tips with a narrow, pointed shape, which is ideal for securely gripping and stabilizing implants during insertion.

Each of the first arm 420 and the second arm 440 also includes a handle (i.e., a first handle 426, a second handle 446) positioned at the corresponding proximal end 422, 442 and an implant interface (i.e., a first implant interface 430, a second implant interface 450) positioned at the corresponding distal end 424, 444.

Similar to the surgical insertion tool 300, each of the first handle 426 and the second handle 446 of the surgical insertion tool 400 is ergonomically formed to fit the natural contour of a hand, featuring a curved, elongated profile that gently tapers toward the middle for a secure grip. Additionally, each of the first implant interface 430 and the second implant interface 450 includes a prong (i.e., a first prong 432, a second prong 452) configured to receive a portion of an implant. Each prong (i.e., the first prong 432, the second prong 452) is shaped to complement the profile of the implant's body or peg, ensuring a secure fit.

Referring to the first arm 420, the proximal end 422 also includes a pair of holes 425 positioned on a first interior side surface 412 of the first arm 420. Each hole 425 extends into the first arm 420 and is configured to receive a corresponding protrusion 445 extending from the second arm 440, as is further detailed below.

Each hole 425 extends perpendicular to a plane P4, which is parallel to the first interior side surface 412 of the first arm 420. This configuration ensures that the protrusions 345 from the second arm 440 align precisely with the holes 425, enabling secure coupling between the two arms 420 and 440.

In this configuration, the holes 425 are positioned on top of one another along the length of the first arm 420. However, the holes 425 may be positioned in any configuration and are not limited to the orientation in the present disclosure.

Referring to the second arm 440, the proximal end 442 of the second arm 440 includes a pair of protrusions 445 extending horizontally from the second interior side surface 416. The pair of protrusions 445 are configured to be inserted into the corresponding holes 425 disposed into the first arm 420. The pair of protrusions 445 extend perpendicularly from the plane P4 that is also parallel to the second interior side surface 416. The pair of protrusions 445 are configured to fit securely into corresponding holes 425 disposed on the first interior side surface 412 of the first arm 420.

As noted above, the surgical insertion tool 400 includes the flexible component 460, which is configured to couple the first arm 420 to the second arm 440. Specifically, the flexible component 460 connects the proximal end 422 of the first arm 420 to the proximal end 442 of the second arm 440. Additionally, the flexible component 460 is configured to bias the first arm 420 and the second arm 440 into an open position, in which the distal ends (i.e., distal end 424 and distal end 444) are spaced apart from each other. This biasing function helps to ensure that the surgical insertion tool 400 remains open by default, allowing for ease of operation and reducing the likelihood of unintended closure.

In some examples, as depicted in FIGS. 6, 7A, and 7B, the flexible component 460 includes a pin 462 and an elastic rod 464. The pin 462 has a first end 466 coupled to the first arm 420, near the proximal end 422, and a second end 468 that is opposite the first end 466. The pin 462 protrudes out of the first interior side surface 412 and is perpendicular to the plane P4, which is parallel to the first interior side surface 412 of the first arm 420. The elastic rod 464 passes perpendicularly through the second end 468 of the pin 462.

The pin 462 is configured to be inserted into a corresponding hole 467 disposed on the second interior side surface 416 of the second arm 440 such that when the pin 462 is inserted, the elastic rod 464 remains positioned perpendicular to the surface of the hole 467. The hole 467 in the second arm 440 constrains the elastic rod’s 464 movement, ensuring that it remains in place while allowing it to deform when force is applied. As the force brings the distal ends 424 and 444 of the arms 420, 440 closer together, the elastic rod 464 deforms—either stretching or compressing—depending on the direction of the applied force. This deformation allows the arms 420, 440 to move while the elastic rod 464 resists the movement, helping to return the arms 420, 440 to their original positions when the force is released.

This deformation enables the arms (i.e., the first arm 420, the second arm 440) to move toward each other while the elastic rod 464 generates a restoring force that urges the arms back to the open position when the applied force is released.

In some examples, the flexible component 460 also includes a knob 469 positioned near the first end 466 of the pin 462 and configured to secure the position of the first arm 420 relative to the second arm 440 by transitioning between a locked and an unlocked state when actuated. For example, the knob 469 prevents the first arm 420 from moving relative to the second arm 440 by engaging a locking mechanism, such as a pin or latch, that secures both arms 420 and 440 in place.

The configuration of the flexible component 460 provides several advantages. The elastic rod 464 ensures smooth and controlled movement of the arms (i.e., the first arm 420, the second arm 440) while maintaining the structural integrity of the surgical insertion tool 400. This configuration also helps to enable repeated cycles of opening and closing without significant wear, making the surgical insertion tool 400 suitable for applications requiring durability and reliability, such as clamping, gripping, or other mechanical operations. Moreover, the perpendicular orientation of the elastic rod 464 through the pin 462 allows for efficient force transfer and minimizes stress on individual components, contributing to the overall lifespan of the tool.

In an example implementation, the surgical insertion tool 400 is configured as a single-piece construction, such that all components are permanently connected. In yet another example implantation, the surgical insertion tool 400 is configured as a multi-piece construction, where the surgical insertion tool 400 can be disassembled.

It should be understood that arrangements described herein are for purposes of example only. As such, those skilled in the art should appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and examples have been disclosed herein, other aspects and examples should be apparent to those having ordinary skill in the art. The various aspects and examples disclosed herein are for purposes of illustration and are not intended to be limiting, with the true scope being indicated by the following claims, along with the full scope of equivalents to which such claims are entitled. It is also to be understood that the terminology used herein is for the purpose of describing particular examples only, and is not intended to be limiting.

Example methods and systems are described herein. It should be understood that the words “example,” “exemplary,” and “illustrative” are used herein to mean “serving as an example, instance, or illustration.” Any example or feature described herein as being an “example,” being “exemplary,” or being “illustrative” is not necessarily to be construed as preferred or advantageous over other examples or features. The examples described herein are not meant to be limiting. It should be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other examples may include more or less of each element shown in a given Figure. Further, some of the illustrated elements may be combined or omitted. Yet further, an example may include elements that are not illustrated in the Figures.

In the following description, numerous specific details are set forth to provide a thorough understanding of the disclosed concepts, which may be practiced without some or all of these particulars. In other instances, details of known devices and/or processes have been omitted to avoid unnecessarily obscuring the disclosure. While some concepts are described in conjunction with specific examples, it should be understood that these examples are not intended to be limiting.

As used herein, “coupled” means associated directly as well as indirectly. For example, a member A may be directly associated with a member B, or may be indirectly associated therewith, e.g., via another member C. It should be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” or “an example” means that one or more feature, structure, or characteristic described in connection with the example is included in at least one implementation. The phrases “one embodiment” or “one example” or “an example” in various places in the specification may or may not be referring to the same example.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The limitations of the following claims are not written in means-plus-function format and are not intended to be interpreted based on 35 U.S.C. § 112(f), unless and until such claim limitations expressly use the phrase “means for” followed by a statement of function void of further structure.

By the term “about,” “approximately,” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, in one embodiment, the term “about” can refer to ± 5% of a given value.

Illustrative, non-exhaustive examples, which may or may not be claimed, of the subject matter according to the present disclosure are provided below.

What is claimed is:

1. A surgical insertion tool comprising:
a first arm including a proximal end and a distal end, the proximal end having a handle and the distal end having an implant interface configured to hold a portion of an implant;
a second arm including a proximal end and a distal end, the proximal end having a handle and the distal end having an implant interface configured to hold a portion of an implant; and
a flexible crossbar configured to couple the first arm to the second arm, the flexible crossbar including a first end coupled at an intermediate point between the proximal end and the distal end of the first arm and a second end coupled at an intermediate point between the proximal end and the distal end of the second arm.

2. The surgical insertion tool of claim 1, wherein the flexible crossbar is configured to bias the distal end of the first arm a first distance away from the distal end of the second arm, and bias the proximal end of the first arm a second distance away from the proximal end of the second arm.

3. The surgical insertion tool of claim 2, wherein the first distance between the distal end of the first arm and the distal end of the second arm is smaller than the second distance between the proximal end of the first arm and the proximal end of the second arm.

4. The surgical insertion tool of claim 1, wherein actuation of the handle at the proximal end of the first arm and the handle at the proximal end of the second arm toward each other moves the implant interface of the first arm away from the implant interface of the second arm.

5. The surgical insertion tool of claim 1, wherein the first arm further comprises a first notch disposed adjacent the distal end of the first arm, and
wherein the second arm further comprises a second notch disposed adjacent to the distal end of the second arm.

6. The surgical insertion tool of claim 5, wherein actuation of the first notch of the first arm toward the second notch of the second arm moves the implant interface of the first arm toward the implant interface of the second arm.

7. The surgical insertion tool of claim 1, wherein the flexible crossbar comprises:
a beam having a first end and a second end;
a first hole positioned near the first end of the beam;
a second hole positioned near the second end of the beam; and
a pair of fastening members,
wherein the first end of the beam is coupled to the first arm by fastening a fastening member of the pair of fastening members through the first hole, and
wherein the second end of the beam is coupled to the second arm by fastening a fastening member of the pair of fastening members through the second hole.

8. The surgical insertion tool of claim 7, wherein each fastening member comprises a bolt and a corresponding nut.

9. The surgical insertion tool of claim 1, wherein the flexible crossbar is configured to control an amount of flexion of the first arm and the second arm by adjusting the pair of fastening members.

10. The surgical insertion tool of claim 1, wherein the flexible crossbar is configured to control an amount of flexion between the first arm and the second arm by adjusting the pair of fastening members.

11. The surgical insertion tool of claim 1, wherein the implant interface of the first arm and the implant interface of the second arm each comprise a groove configured to fit a profile of the implant.

12. The surgical insertion tool of claim 11, wherein the groove of the first arm and the groove of the second arm each comprise a friction surface.

13. The surgical insertion tool of claim 12, wherein the friction surface is made of rubber material.

14. The surgical insertion tool of claim 1, wherein the handle comprises a tab with a plurality of ridges.

15. The surgical insertion tool of claim 1, wherein the first arm, the second arm, and the flexible crossbar are manufactured as one single part.

16. The surgical insertion tool of claim 1, wherein a width of the first arm gradually tapers from the proximal end to the distal end, and wherein a width of the second arm gradually tapers from the proximal end to the distal end.

17. The surgical insertion tool of claim 1, wherein the distal end of the first arm and the distal end of the second arm each comprise a tapered tip.

18. A surgical insertion tool comprising:
a first arm including a proximal end, a distal end opposite the proximal end, and an inner side surface extending from the proximal end to the distal end, the proximal end having a pair of holes disposed on the inner side surface and extending into the first arm, the distal end having a first implant interface configured to interface with a portion of an implant;
a second arm including a proximal end, a distal end opposite the proximal end, and an inner side surface extending from the proximal end to the distal end, the proximal end having a pair of protrusions extending horizontally from the inner side surface and configured to be inserted into the corresponding pair of protrusions of the first arm to connect the first arm to the second arm, the distal end having a second implant interface configured to interface with a portion of an implant; and
a flexible component configured to couple the proximal end of the first arm to the proximal end of the second arm.

19. The surgical insertion tool of claim 18, wherein the first implant interface and the second implant interface each comprise a prong.

20. The surgical insertion tool of claim 18, wherein the first implant interface and the second implant interface each comprise a friction surface.

21. The surgical insertion tool of claim 20, wherein the friction surface is made of rubber material.

22. The surgical insertion tool of claim 18, wherein a width of the first arm gradually tapers from the proximal end to the distal end, and wherein a width of the second arm gradually tapers from the proximal end to the distal end.

23. The surgical insertion tool of claim 18, wherein the distal end of the first arm and the distal end of the second arm each comprise a tapered tip.

24. The surgical insertion tool of claim 18, wherein the flexible component is a tension spring having a first end positioned within the first arm, near the proximal end, and a second end positioned within the second arm, near the proximal end.

25. The surgical insertion tool of claim 18, wherein the flexible component comprises a pin having a first end and a second end that is opposite the first end, and a helical spring positioned around the pin,
wherein the first end is positioned within the first arm, near the proximal end, and the second end is positioned within the second arm, near the proximal end, and
wherein the helical spring deforms as the distal end of the first arm and the distal end of the second arm move toward one another.

26. The surgical insertion tool of claim 18, wherein the flexible component comprises a pin having a first end and a second end that is opposite the first end, and an elastic rod that intersects perpendicularly through the second end of the pin,
wherein the first end is positioned within the first arm, near the proximal end, and the second end is positioned within the second arm, near the proximal end, and
wherein the elastic rod deforms as the distal end of the first arm and the distal end of the second arm move toward one another.

27. The surgical insertion tool of claim 24, wherein the flexible component includes a knob configured to secure a position of the first arm relative to the second arm by transitioning between a locked and an unlocked state when actuated.

28. The surgical insertion tool of claim 18, wherein actuation of the distal end of the first arm toward the distal end of the second arm moves the first implant interface toward the second implant interface to a closed position.

29. The surgical insertion tool of claim 18, wherein the flexible component is configured to control an amount of flexion between the first arm and the second arm.
